(19) United States
(12) Patent Application Publication
(10) Pub. No.: US 2009/0131527 A1
(43) Pub. Date: May 21, 2009

(54) NOVEL ANTHRANILIC ACID DERIVATIVES AND CHLORIDE CHANNEL BLOCKING AGENT CONTAINING THE SAME

(75) Inventors: Changjoon Justin Lee, Seoul (KR); Eun-Joo Roh, Seoul (KR); Soo-Jin Oh, Seoul (KR); Jung-Hwan Park, Seoul (KR); Jae-Kyun Lee, Seoul (KR)

Correspondence Address: LEXYOUME IP GROUP, LLC, 5180 PARKSTONE DRIVE, SUITE 175, CHANTILLY, VA, 20151, US

(73) Assignee: Korea Institute of Science and Technology, Seoul (KR)

(21) Appl. No.: 12/022,405

(22) Filed: Jan. 30, 2008

(30) Foreign Application Priority Data
Nov. 19, 2007 (KR) 10-2007-0117886

(51) Int. Cl. A61K 31/196; C07C 229/58; C07C 227/18; C12Q 1/02; A61P 29/00; A61P 35/00

(52) U.S. Cl. 514/567; 562/456; 562/435; 435/29

(57) ABSTRACT

The present invention relates to novel anthranilic acid derivatives represented by Chemical Formula I, and a chloride channel blocking agent containing the anthranilic acid derivative or its pharmacologically acceptable salts as an active ingredient. In another aspect, the present invention relates to a method of accurately and efficiently detecting the intracellular chloride channel inhibition and method of screening a chloride channel blocking agent.

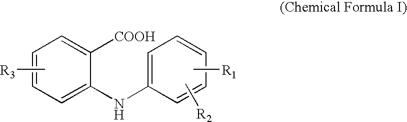

### CROSS-REFERENCE TO RELATED APPLICATION

This application claims priority to and the benefit of Korean Patent Application No. 10-2007-0117886 filed in the Korean Intellectual Property Office on Nov. 19, 2007, the entire contents of which are incorporated herein by reference.

### BACKGROUND OF THE INVENTION

(a) Field of the Invention

The present invention relates to novel anthranilic acid derivatives represented by Chemical Formula I, and a chloride channel blocking agent containing the anthranilic acid derivative or its pharmacologically acceptable salts as an active ingredient. In another aspect, the present invention relates to a method of accurately and efficiently detecting the intracellular chloride channel inhibition and method of screening a chloride channel blocking agent.

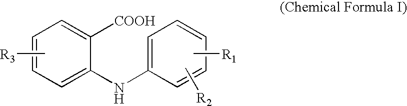

(b) Description of the Related Art

A chloride channel is found in almost every cell encompassing bacteria to mammals. A chloride channel induced from these diverse cell types has a characteristic of being activated by a calcium ion concentration of 0.2 to 5 mM in the cytoplasm. In frog eggs, where the existence of chloride channels was first identified in 1980, a calcium sensitive chloride channel opens by an elevated calcium ion concentration at the time of fertilization, causing depolarization of the cell membrane, which in turn may prevent the entry of sperm cells. The existence of calcium dependent chloride channels has been subsequently proven in various nerve cells and others cells.

The chloride channel has a variety of functions depending on the derived cells, and the major functions include secretion of epithelium, membrane excitation in the myocardial and nerve cells, conduction path of the smell sense, controlling the heart pulse rate, and controlling the reactivity in photoreceptors. Even though research on calcium-dependent chloride channels has more than a 20-year history, since 1980, research on the physiological function and controlling mechanism is still lacking, and the main reason is due to the absence of an appropriate calcium channel blocking agent. Therefore, it is urgent to develop a chloride channel blocking agent with efficiency and selectivity.

### SUMMARY OF THE INVENTION

To satisfy the above requirements, the present inventors have prepared anthranilic acid derivatives having prominent inhibitory activity against chloride channels as a result of study to develop a chloride channel blocking agent with efficiency and selectivity, to complete the present invention.

Therefore, an embodiment of the present invention provides novel anthranilic acid derivatives and a preparation method thereof.

Another embodiment of the present invention provides a chloride channel blocking agent including the anthranilic acid derivatives or their pharmacologically acceptable salts as an active ingredient.

Still another embodiment of the present invention provides a method of determining the inhibition of intracellular chloride channels and a method to screening chloride channel blocking agents.

### BRIEF DESCRIPTION OF THE DRAWINGS

FIG. 1 is a graph showing the activation of chloride channel by Ca²⁺ in a Xenopus Laevis oocyte, wherein

1a shows the current induced in an ionomycin treated oocyte in a dose-dependent manner by extracellular Ca²⁺,

1b shows EC₅₀ and dose response of Ca²⁺ obtained in the above 1a,

1c is a graph recording the current obtained after the treatment of 10 μM ionomycin for 30 min. without the treatment of thapsigargin,

1d is a graph recording the current obtained after the treatment of 10 μM ionomycin for 30 min. and subsequent treatment of 1 μM thapsigargin for 90 min., and

1e to 1i are graphs comparing the results with (+TG) and without (−TG) thapsigargin treatments.

FIG. 2 shows the effect of existing blocking agents of the Cl⁻ channel activated by Ca²⁺, wherein

2a is a graph recording the current changes in the Cl⁻ channel activated by Ca²⁺ before and during the application of flufenamic acid,

2b is a graph showing the dose response relationship in the inhibition of the Cl⁻ channel activated by Ca²⁺ by flufenamic acid, wherein n is the number of oocytes, and

2c shows the IC₅₀ values of commercial blocking agents for the Cl⁻ channel activated by Ca²⁺.

FIG. 3 shows the effect of anthranilic acid derivatives on the Cl⁻ channel activated by Ca²⁺, wherein

3a is a graph recording the current changes in the Cl⁻ channel activated by Ca²⁺ before and during the application of N-(4-chlorophenyl)-anthranilic acid,

3b is a graph showing the dose response relationship in the inhibition of the Cl⁻ channel activated by Ca²⁺ by N-(4-chlorophenyl)-anthranilic acid, wherein n is the number of oocytes, and

3c shows the IC₅₀ values of anthranilic acid derivatives for the Cl⁻ channel activated by Ca²⁺.

FIG. 4 is a graph showing the effect of a calcium channel blocking agent to the Cl⁻ channel activated by Ca²⁺ according to the position of functional groups in the phenyl ring, wherein

4a shows the comparison between chemical structure, IC₅₀, and dose response relationship according to the position of a nitro group in the phenyl ring of nitrophenylanthranilic acid, and

4b shows the comparison between chemical structure, IC₅₀, and dose response relationship according to the position of a trifluoromethyl group in the phenyl ring of trifluoromethylphenylflufenamic acid derivatives.

### DETAILED DESCRIPTION OF THE EMBODIMENTS

In the following detailed description, only certain exemplary embodiments of the present invention have been shown and described, simply by way of illustration.

As those skilled in the art would realize, the described embodiments may be modified in various ways, all without departing from the spirit or scope of the present invention.

Accordingly, the drawings and description are to be regarded as illustrative in nature and not restrictive.

Like reference numerals designate like elements throughout the specification.

In addition, unless explicitly described to the contrary, the word “comprise”, and variations such as “comprises” or “comprising”, will be understood to imply the inclusion of stated elements, but not the exclusion of any other elements.

The present invention relates to novel anthranilic acid derivatives, a chloride channel blocking agent containing the anthranilic acid derivative or their pharmacologically acceptable salts as an active ingredient, a detection method to detect the inhibition of the intracellular chloride channel accurately and efficiently, and a screening method of the chloride channel blocking agent using the same.

Anthranilic acid is one of aromatic amino acids, and is also known as aminobenzoic acid. The present invention was completed by the preparation of a variety of anthranilic acid derivatives and confirming that these compounds are useful as chloride channel blocking agents.

The present invention relates to novel anthranilic acid derivatives represented by Chemical Formula I:

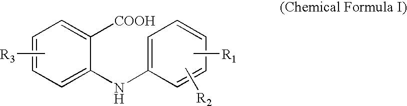

wherein R₁ and R₂, respectively, are independently selected from the group consisting of a hydrogen atom, a halogen atom, a linear, branched, or cyclic alkyl group having 5 to 16 carbon atoms, a linear or branched substituted alkyl group having 1 to 16 carbon atoms, a substituted or non-substituted aryl groups having 6 to 12 carbon atoms, an alkoxy group having 2 to 8 carbon atoms, a substituted alkoxy group having 1 to 8 carbon atoms, and a nitro group, and R₁ and R₂ are not hydrogen at the same time.

When the above alkyl or alkoxy group is a substituted alkyl or substituted alkoxy group, one or more hydrogen atoms of an alkyl or alkoxy compound may be substituted independently with one selected from the group consisting of a halogen atom and a phenyl group. When the above aryl group is substituted, one or more hydrogen atoms of an aryl compound may be substituted independently with one or more selected from the group consisting of a halogen atom and an alkyl group having 1 to 16 carbon atoms. In the present invention, the substances that may be used as the above R₁ and R₂ may be selected from the substances including racemic mixtures and chiral compounds. R₃ is a hydrogen atom or nitro group. When R₃ is a hydrogen atom, the following are excluded from the compounds used as the chloride channel blocking agent in the present invention:

one of R₁ and R₂ being 3-trifluoromethyl and the other being a hydrogen atom;

one of R₁ and R₂ being 2-trifluoromethyl and the other being 3-trifluoromethyl;

one of R₁ and R₂ being 4-fluoro, 4-chloro-, or 4-bromo, and the other being a hydrogen atom;

one of R₁ and R₂ being 2-chloro and the other being 3-chloro or 4-chloro; and

one of R₁ and R₂ being 3-chloro and the other being 4-chloro or 5-chloro.

For example, the above R₁ and R₂, respectively, are independently selected from the group consisting of a hydrogen atom, a pentyl group, a hexyl group, a heptyl group, an octyl group, a decyl group, a dodecyl group, a tetradecyl group, a hexadecyl group, a cyclohexyl group, a phenyl group, a benzyl group, a racemic-alpha methyl-benzyl group, a chiral-alpha methyl-benzyl group, a trifluoromethyl group, a methoxyl group, an ethoxyl group, a butyloxy group, a pentyloxy group, a hexyloxy group, a trifluoromethyloxy group, and a nitro group (with the proviso that R1 and R2 cannot be hydrogen atoms at the same time), or R₁ and R₂ are the same or different halogen atoms. The above substituted aryl group may be a compound wherein one or more hydrogen atoms in the aryl group are substituted with one or more selected from the group consisting of 2,3-dichloro, 2,4-dichloro, 2,5-dichloro, 2,6-dichloro, 3,4-dichloro, 3,5-dichloro, 2,4,5-trichloro, 2,4,6-trichloro, 3,4,5-trichloro, and 2,3,5,6-tetrachloro.

In a preferred embodiment, the compound represented by Chemical Formula I may be selected from the group consisting of decylphenyl anthranilic acid, hexaoxyphenyl anthranilic acid, trifluoromethylphenyl anthranilic acid, nitrophenyl anthranilic acid, 4-fluoro-3-trifluoromethylphenyl anthranilic acid, 2,4-ditrifluoromethylphenyl anthranilic acid, 5-nitro-N-(4-trifluoromethylphenyl)anthranilic acid, and 5-nitro-N-(4-nitrophenyl)anthranilic acid.

In another aspect, the present invention relates to a preparation method of the compounds represented by following Chemical Formula I, including the steps of:

reacting the compound represented by Chemical Formula 1 with chlorotrimethylsilane in the presence of methanol or ethanol, or esterifying by using methanol or ethanol in the presence of an acid catalyst, to prepare a compound represented by Chemical Formula 2 (Step 1);

reacting the obtained compound represented by Chemical Formula 2 with an aniline compound represented by Chemical Formula 3, to prepare a compound represented by Chemical Formula 4 (Step 2); and

reacting the compound represented by Chemical Formula 4 with one or more selected from the group consisting of alkali metal hydroxides and alkali earth metal hydroxides, to obtain the compound represented by Chemical Formula I (Step 3),

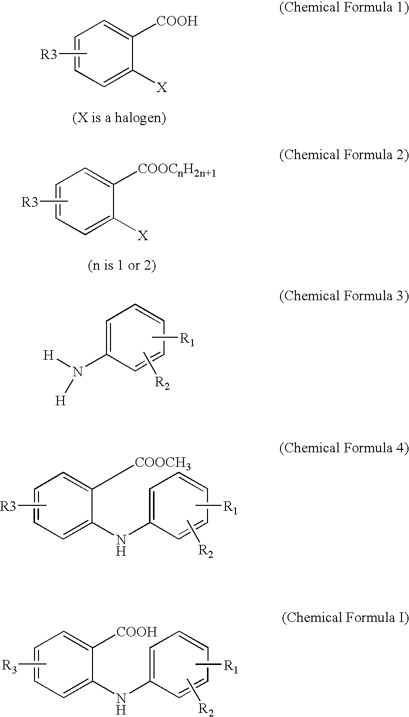

wherein the identifications of R₁, R₂, and R₃ are as defined in the above Chemical Formula I.

In step 1, there are no specific limitations as to the esterification method. Even though Fischer esterification using alcohol in the presence of an acid catalyst is mainly used industrially, an esterification method with chlorotrimethylsilane may be used in consideration of reactivity and mild conditions (room temperature) in the detailed embodiments of the present invention. Chlorotrimethylsilane used in step 1 is a chemical that may esterify a carboxy group of aryl compounds in the above Chemical Formula 1, and it is preferable to introduce an methyl or ethyl group while bearing in mind that they are easily hydrolyzed even though it is possible to synthesize esters with various alkyl groups depending on the alcohols used as reactants or solvents. Therefore, a suitable alcohol may be selected and used depending on the alkyl groups that one desires to introduce, and it is preferable to use methanol or ethanol. Also, step 1 may be carried out by the esterification method by using a suitable alcohol in the presence of an acid catalyst, where the above alcohol may be methanol or ethanol. There are no specific limitations as to the kinds of the above acid catalysts, and for example, they may be selected from the group consisting of one or more of sulfuric acid (H₂SO₄), hydrochloric acid (HCl), and phosphoric acid (H₃PO₄). Also in the above step 1, chemicals may be obtained by refluxing for 6 to 12 hours in the presence of an alcohol solvent.

Step 2 may be carried out by complex formation with a suitable ligand in the presence of a catalyst. It is important to form the complex between the metal catalyst and the ligand first, and it is preferable to reflux for 24 to 48 hours in the presence of a suitable solvent. The catalysts that may be used in the above step 2 include a catalyst that may form a bond between an amino group of aniline derivatives of the above Chemical Formula 3 at the halogen atom (X) and the chemical of the above Chemical Formula 2, and palladium catalysts exemplified as palladium acetate (Pd(OAc)₂), tris(dibenzylideneacetone)dipalladium ((Pd₂(dba)₃), and tris(dibenzylideneacetone)dipalladium·chloroform complex (Pd₂(dba)₃ CHCl₃) may be used. Also, bisphosphine chemicals may be used as the above ligand, and for example, 1,1′-diphenylphosphinoferrocene (dppf) or 2,2′-bis(diphenylphosphino)-1,1′-binaphthalene (BINAP) may be used, and cesium carbonate (Cs₂CO₃) may be used as a base.

Also, any solvent that may dissolve the reactant aryl compounds may be used as the above organic solvent, and for example, it may be one or more selected from the group consisting of 1,4-dioxane and toluene, and it is more suitable to use toluene.

The metal compounds used in the above step 3 may be any metal compounds that may produce a carboxy group when reacting with the methyl ester group of the above Chemical Formula 4, and are preferably one or more selected from the group consisting of alkali metal hydroxides and alkali earth metal hydroxides, and for example, may be lithium hydroxide (LiOH), potassium hydroxide (KOH), and/or sodium hydroxide (NaOH).

Also, step 3 may be performed in the presence of a suitable solvent, and the above solvent may be one or more selected from the group consisting of water and methanol. When a mixture of water, tetrahydrofuran, and methanol is used, the highest reactivity is obtained when the mixing ratio between water:tetrahydrofuran:methanol is 3:0.5˜1.5:0.5˜1.5, and preferably 3:1:1, in weight ratio. The above step 3 may be performed at the temperature range of 15 and 30° C., and preferably at room temperature, and it is preferable to perform the reaction for 12 to 24 hours to continue the reaction sufficiently.

In another aspect, the present invention provides a chloride channel blocking agent including one or more of the following compounds having the structure of Chemical Formula I-1 or their pharmacologically acceptable salts as an active ingredient.

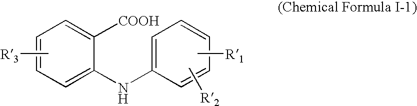

In the above formulas, R′₁, and R′₂ may be independently selected from the group consisting of a hydrogen atom, a halogen atom, a linear, branched, or cyclic substituted or non-substituted alkyl group having 1 to 16 carbon atoms, a substituted or non-substituted aryl group having 6 to 12 carbon atoms, a substituted or non-substituted alkoxy group having 1 to 8 carbon atoms, and a nitro group. In case the above alkyl or alkoxy group is substituted, one or more hydrogen atoms of an alkyl or alkoxy group may be substituted independently with one or more selected from the group consisting of halogen atoms and a phenyl group. In case the above aryl group is substituted, one or more hydrogen atoms of an aryl group may be independently substituted with one or more selected from the group consisting of a halogen atoms and an alkyl group having 1 to 6 carbon atoms. R1 and R2 cannot be hydrogen atoms simultaneously. In an embodiment of the present invention, the substances used as the above R′₁ and R′₂ may be selected from substances including racemic mixtures and chiral compounds. The R′₃ may be a hydrogen atom or a nitro group.

For example, the R′₁, and R′₂ may be independently selected from the group consisting of hydrogen, fluorine, chlorine, bromine, iodine, a methyl group, an ethyl group, a propyl group, an isopropyl group, a butyl group, a sec-butyl group, a tert-butyl group, a pentyl group, a hexyl group, a heptyl group, an octyl group, a decyl group, a dodecyl group, a tetradecyl group, a hexadecyl group, a cyclohexyl group, a phenyl group, a benzyl group, a racemic-alpha methyl-benzyl group, a chiral-alpha a methyl-benzyl group, a trifluoromethyl group, a methoxyl group, an ethoxyl group, a butyloxy group, a pentyloxy group, a hexyloxy group, a trifluoromethyloxy group, and a nitro group. The substituted alkyl group may be a compound wherein one or more hydrogen atoms in the aryl group may be substituted with one or more selected from the group consisting of 2,3-dichloro, 2,4-dichloro, 2,5-dichloro, 2,6-dichloro, 3,4-dichloro, 3,5-dichloro, 2,4,5-trichloro, 2,4,6-trichloro, 3,4,5-trichloro, and 2,3,5,6-tetrachloro groups.

In case R′₃ is a hydrogen atom, however, the following cases are excluded from the compounds used as the chloride channel blocking agent in the present invention:

when one is 3-trifluoromethyl and the other is a hydrogen atom between R′₁ and R′₂; and

when one is 2-trifluoromethyl and the other is 3-trifluoromethyl between R′₁ and R′₂.

In a preferred embodiments, the compounds of Chemical Formula I-1 may be independently selected from the group consisting of fluorophenyl anthranilic acid, chlorophenyl anthranilic acid, methylphenyl anthranilic acid, isopropylphenyl anthranilic acid, tert-butylphenyl anthranilic acid, decylphenyl anthranilic acid, methoxylphenyl anthranilic acid, hexaoxyphenyl anthranilic acid, trifluoromethylphenyl anthranilic acid, trifluoromethylphenyl anthranilic acid, nitrophenyl anthranilic acid, 4-fluoro-3-trifluoromethylphenyl anthranilic acid, 2,4-ditrifluoromethylphenyl anthranilic acid, 5-nitro-N-(4-trifluoromethylphenyl)anthranilic acid, and 5-nitro-N-(4-nitrophenyl)anthranilic acid. In the detailed description of the especially preferred embodiments, the chemicals in the above Formula I-1 may be independently selected from the group consisting of N-(4-trifluoromethylphenyl)anthranilic acid, N-(4-chlorophenyl)anthranilic acid, N-(4-fluoro-3-trifluoromethylphenyl)anthranilic acid, 5-nitro-N-(4-nitrophenyl)anthranilic acid, N-(4-nitrophenyl)anthranilic acid, N-(4-tert-butylphenyl)anthranilic acid, N-(2-trifluoromethylphenyl)anthranilic acid, and N-(3-nitrophenyl)anthranilic acid.

A chloride channel is found in a wide variety of animal cells encompassing bacteria to mammals, and its opening and closing are controlled by the concentration of intracellular calcium ion. That is, the chloride channel opens when the intracellular calcium ions increase. In the present invention, it was confirmed that the activation of the chloride channel may be blocked by treating frog ovum cells, a representative animal cell model of the chloride channel, with the compound of Chemical Formula I (see Example 2 below).

The importance of the calcium-dependent chloride channel has been recognized due to various physiological functions, but it has been difficult to develop the blocking agents with selectivity since there are a few subtypes. Anthranilic acid derivatives according to the present invention are excellent chloride channel blocking agents with high efficiency and selectivity.

As described above, the chloride channel has a variety of physiological functions including secretion of epithelium, membrane excitation in the myocardial and nerve cells, in the conduction path of the smell sense, controlling the pulse rate, and controlling reactivity in photoreceptors. When the chloride channel is over-activated or when one becomes sensitive to the chloride channel depending on personal characteristics of the patient, many diseases, for instance fibroma, inflammation, and dystrophy may occur. Main diseases caused by the above include cystic fibrosis, chronic or acute bronchitis, and vitelliform macular dystrophy in the pancreas, lung, or airway (refer to Chloride channels and cystic fibrosis of the pancreas. Biosci Rep. December 1995;15(6):531-41; Interaction between calcium-activated chloride channels and the cystic fibrosis transmembrane conductance regulator. Pflugers Arch 1999;438:635-641; The genesis of cystic fibrosis lung disease. J Clin Invest 1999;103:309-312; Increased expression of the calcium-activated chloride channel hCLCA1 in airways of patients with obstructive chronic bronchitis. Respir J. April 2005;12(3):143-6; New VMD2 gene mutations identified in patients affected by Best vitelliform macular dystrophy. J Med Genet. March 2007;44(3):e70. Epub Feb. 7, 2007. The above literature is included in the present embodiment as references).

Therefore, diseases caused by the chloride channel as above may be prevented and/or treated by administration of the chloride channel blocking agent according to the present invention to patients with hyperactive chloride channels or to chloride channel sensitive patients.

Another aspect of the present invention is to provide a composition including a compound represented by Chemical Formula I-1 or its pharmacologically acceptable salts as an active ingredient to prevent and/or to treat one or more diseases selected from the group consisting of fibroma, inflammation, and dystrophy. In an embodiment of the present invention, the composition may be used to prevent and/or treat one or more diseases selected from the group consisting of cystofibroma of the pancreas, lung, or trachea, chronic or acute bronchitis, and vitelliform macular dystrophy. The chloride channel blocking agent and the composition according to the present invention to prevent and/or to treat one or more of the diseases caused by the calcium channel may contain the compound represented by Chemical Formula I-1 or its pharmacologically acceptable salts as an active ingredient in the amount of 0.001 to 100 weight %, and it is possible to be administered orally or non-orally. The daily dose of the above chloride channel blocking agent and the composition to prevent and/or treat one or more of the diseases caused by the calcium channel is in a range of 0.001 to 100 mg/kg (weight of the patient), and preferably in a range of 0.1 to 50 mg/kg (weight of the patient), based on the compound represented by Chemical Formula I-1 or its pharmacologically acceptable salts as an active ingredient.

In an embodiment of the present invention, the patient may be an animal or animal cells, is preferably mammals, and is more preferably human beings.

In an embodiment of the present invention, the chloride channel blocking agent or the composition to prevent and/or to treat one or more diseases caused by the calcium channel may contain the compound represented by Chemical Formula I-1 or its pharmacologically acceptable salts as an active ingredient, together with one or more additives, excipients, carriers, buffers, diluents, and/or conventional pharmaceutical adjuvants.

In a preferable aspect, the present invention provides a pharmaceutical composition containing the compound according to the present invention or its pharmaceutically acceptable salts or derivatives and optionally additionally containing components known and used in the relevant field to treat and/or prevent such diseases, together with one or more pharmaceutically acceptable carriers.

The wording “a material is pharmaceutically acceptable” as mentioned above means that the material is able to be mixed with other components and is harmless to the patients who receive the administration.

The blocking agent or the composition according to the present invention may be formulated so as to be suitable for rectal, tracheal, nasal, intra-lung, local (including buccal and sublingual), transdermal, vaginal, or non-oral (dermal, subcutaneous, intramuscular, intraperitoneal, intravenous, intraarterial, intracranial, and intraocular) administration, and is suitable for inhalation or insufflation including the administration of dispersion or liquid aerogel or is suitable for a sustained release system. Suitable examples of the sustained release system may include a semipermeable matrix of a hydrophobic solid polymer containing the compound of the present invention, wherein the matrix may, for example, be a molded form of film or microcapsules.

In yet another aspect, the present invention provides a method to measure the inhibition of chloride channels in animal cells by using a two-electrode voltage clamp method. The voltage clamp method is a method to measure and analyze the changes in the cell membrane potential induced by a specific ion channel existing on a cell membrane or signal transducing material. The two-electrode voltage clamp method is a method to measure and analyze the changes in the cell membrane potential by two electrodes, one to clamp the cell membrane potential and the other to measure current among the voltage clamp methods. The two-electrode voltage clamp method may be readily understood by a person having ordinary skill in the art to which the present invention belongs.

More preferably, the method to measure the inhibition of chloride channel includes the steps of:

preparing a sample;

treating the sample with calcium;

treating the sample with ionomycin;

treating the ionomycin-treated sample with thapsigargin;

contacting two microelectrodes filled with intracellular solution containing chelerythrine with the ionomycin and thapsigargin treated sample; and

measuring the current generated from the electrodes.

The above samples may be any animal cells, and may be representatively frog ovum cells. The above ionomycin has a function of a calcium ion channel (calcium ionophore), and the treatment concentration may be in the range of 0.1 to 100 μM, preferably 1 to 50 μM, and more preferably 5 to 30 μM. Also, the above thapsigargin is one of the Ca²⁺-ATPase inhibitors, and the treatment concentration may be in the range of 0.1 to 10 μM, preferably 0.75 to 1.25 μM, and more preferably approximately 1 μM. Further, the above chelerythrine is a kind of protein kinase inhibitor, and the treatment concentration may be in the range of 0.1 to 10 μM, preferably 0.75 to 1.25 μM, and more preferably approximately 1 μM. The above ranges may be experimentally obtained as the optimum conditions to control the calcium receptor and to obtain accurate results. The present invention may be characterized by measuring the inhibition of chloride channels more sensitively and accurately by treating the above three materials with the above treatment concentrations in order of precedence, and by measuring the produced current.

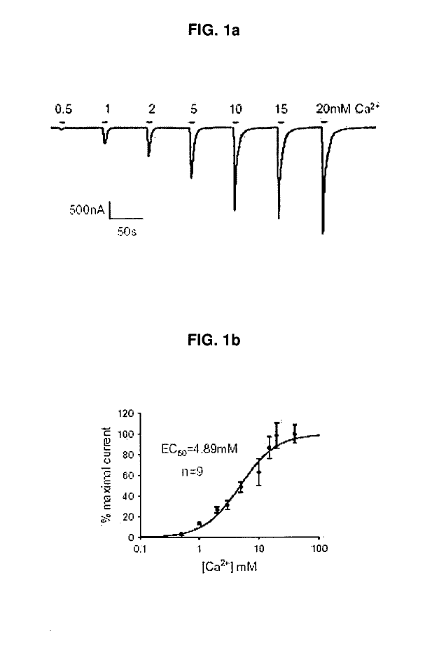

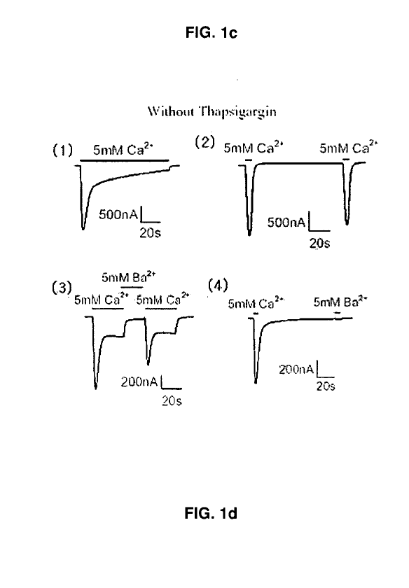

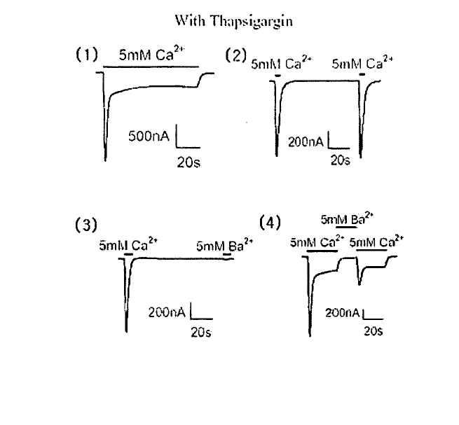

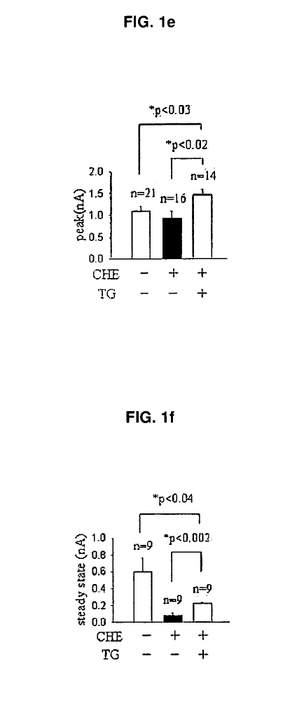

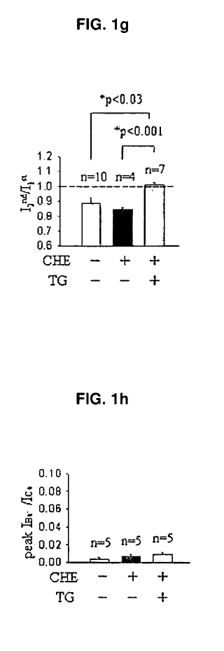

When the sample is treated with calcium ions and in turn with ionomycin, calcium is permeated through ionopores, and the permeated calcium activates the chloride channel and opens the chloride channel and produces the current inside the cells resulting in the current changes. If the current inside the cells does not change or decreases even though the condition is set to permeate calcium into the cells by the treatment with calcium and ionomycin, it means that the chloride channel is blocked. Therefore, it may be determined that the chloride channel is blocked in case the intracellular current production decreases or does not change by performing the above method to test the above chloride channel inhibition. In this test, there is no specific limitation as to the amount of calcium for the treatment, but it is possible to obtain a preferable amount of calcium for the treatment from an experiment to determine EC₅₀ values of calcium for the calcium dependent chloride channel for more efficient experimentation, and the obtained preferred amount of calcium treatment is in the range of 2 to 10 mM, and more preferably approximately 5 mM per cell (refer to FIG. 1b).

There has been no example in the past to utilize the above two-electrode voltage clamp method in the measurement of chloride channel activity, and it is advantageous to increase the accuracy of the measurement considerably by measuring the activity of the chloride channel by using the two-electrode voltage clamp method according to the above specific protocol in the present invention.

In another aspect, the present invention provides a screening method of the chloride channel blocking agent by utilizing the above method of measurement.

More specifically, the screening method of the chloride channel blocking agent according to the present invention includes the steps of:

treating samples with calcium;

treating some of the samples with a candidate material, to prepare candidate material treated samples and untreated samples;

treating the candidate material treated samples and untreated samples with ionomycin and then thapsigargin;

contacting two microelectrodes filled with intracellular solution containing chelerythrine with the samples; and

determining the candidate material as a chloride channel blocking agent when the induced current of the candidate material treated samples is decreased or not changed compared with that of untreated samples.

The above samples and the treatment concentration of the treatment materials are the same as described above.

The importance of the calcium-dependent chlorides has been recognized due to various physiological functions, but the molecular mechanism cannot be elucidated due to the lack of appropriate inhibitors. Also, it has been difficult to develop the blocking agents with selectivity since there are a few subtypes. Under these circumstances, it is possible to develop a chloride channel blocking agent with high efficiency and selectivity by using the screening method according to the present invention.

The importance of the calcium-dependent chlorides has been recognized due to various physiological functions, but the molecular mechanism cannot be elucidated due to the lack of appropriate inhibitors. Also, it has been difficult to develop the blocking agents with selectivity since there are a few subtypes. Under these circumstances, it is expected to develop a chloride channel blocking agent with higher efficiency and selectivity by using the anthranilic acid derivatives proposed by the present invention as a basis for discovery.

The present invention is further explained in more detail with reference to the following examples. These examples, however, should not be interpreted as limiting the scope of the present invention in any manner.

### EXAMPLES

### Example 1

### Preparation of Anthranilic Acid Derivatives 1 (R₃:H)

Preparation of anthranilic acid derivatives in the present invention was carried out by the reaction formulas shown below.

### 1-1. Preparation of 2-bromobenzoic acid methyl ester

Bromobenzoic acid methyl ester was prepared according to the Reaction Formula 1 shown below (yield: 92%):

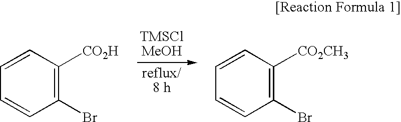

Rf=0.72(EA/n-Hex=1:2)

¹H NMR (400 MHz, CDCl₃)=3.93 (s, 3H), 7.29-7.37 (m, 2H), 7.65 (dd, J=1.58 Hz and 1.54 Hz, 1H), 7.78 (dd, J=2.04 Hz and 2.08 Hz, 1H) ¹³C NMR (100 MHz, CDCl₃)=52.36, 121.53, 127.05, 131.19, 132.01, 132.46, 134.22, 166.50

### 1-2. Preparation of N-aryl anthranilic acid methyl cellulose

N-aryl anthranilic acid methyl cellulose was prepared according to the following Reaction Formula 2 by using the chemicals prepared in the above Example 1-1:

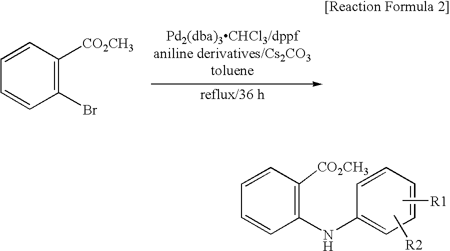

In the above formula, R₁ and R₂, respectively, are independently selected from the group consisting of a hydrogen atom, a halogen atom, a linear or branched substituted or non-substituted alkyl group having 1 to 16 carbon atoms, a substituted or non-substituted aryl group having a number of carbon atoms ranging between 6 and 12, a substituted or non-substituted alkoxy group having 1 to 8 carbon atoms, and a nitro group.

1-3. Preparation of N-aryl anthranilic acid

N-aryl anthranilic acid was prepared according to the following Reaction Formula 3 by using the chemicals prepared in the above Example 1-2:

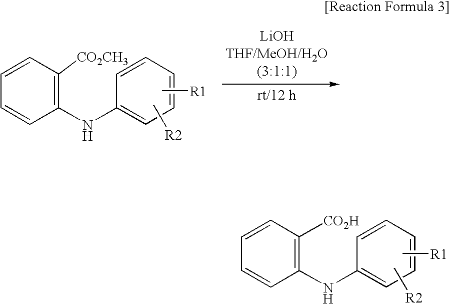

In the above formula, R₁ and R₂, respectively, are independently selected from the group consisting of a hydrogen atom, a halogen atom, a linear or branched substituted or non-substituted alkyl group having 1 to 16 carbon atoms, a substituted or non-substituted aryl group having 6-12 carbon atoms, a substituted or non-substituted alkoxy group having 1 to 8 carbon atoms, and a nitro group.

The below chemicals were prepared according to the above Example 1-1˜1-3:

### N-Phenylanthranilic acid

Yield=82%

White Solid

¹H NMR (400 MHz, CDCl₃)=6.75 (dd, J=7.11 Hz and 7.09 Hz, 1H), 7.13 (quintet, 1H), 7.21-7.39 (m, 6H), 8.05 (d, J=8.04 Hz, 1H), 9.31 (bs, 1H); 13C NMR (100 MHz, CDCl3)=110.37, 114.02, 117.17, 123.14, 124.10, 129.43, 132.61, 135.23, 140.30, 148.92, 173.84

### N-(4-Fluorophenyl)anthranilic acid

Yield=67%

White Powder

¹H NMR (400 MHz, CDCl₃)=6.75 (quintet, 1H), 7.01-7.10 (m, 3H), 7.21-7.39 (m, 6H), 8.05 (d, J=8.04 Hz, 1H), 9.31 (bs, 1H); 13C NMR (100 MHz, CDCl3)=110.37, 114.02, 117.17, 123.14, 124.10, 129.43, 132.61, 135.23, 140.30, 148.92, 173.84

### N-(4-Chlorophenyl)anthranilic acid

Yield=80%

White Powder

¹H NMR (400 MHz, CDCl₃)=6.79 (t, J=7.56 Hz, 1H), 7.16-7.21 (m, 3H), 7.32-7.39 (m, 3H), 8.05 (dd, J=1.43 Hz and 1.42 Hz, 1H), 9.26 (bs, 1H); 13C NMR (100 MHz, CDCl3)=110.66, 113.96, 117.65, 124.25, 129.03, 129.49, 132.67, 135.35, 138.95, 148.49, 173.62

### N-(4-Methylphenyl)anthranilic acid

Yield=74%

Pale Brown Crystal

¹H NMR (400 MHz, CDCl₃)=2.35 (s, 3H), 6.70 (dd, J=7.23 Hz and 7.23 Hz, 1H), 7.11-7.19 (m, 5H), 7.31 (quintet, 1H), 8.03 (dd, J=1.01 Hz and 1.03 Hz, 1H), 9.22 (bs, 1H); 13C NMR (100 MHz, CDCl3)=20.91, 109.89, 113.77, 116.68, 123.80, 130.00, 132.56, 134.06, 135.21, 137.51, 149.57, 174.01

### N-(4-Isopropylphenyl)anthranilic acid

Yield=77%

White Powder

¹H NMR (400 MHz, CDCl₃)=1.27 (d, J=6.96 Hz, 6H), 2.92 (quintet, 1H), 6.72 (quintet, 1H), 7.16-7.33 (m, 6H), 8.03 (dd, J=1.59 Hz and 1.58 Hz, 1H), 9.26 (bs, 1H); 13C NMR (100 MHz, CDCl3)=24.08, 33.63, 109.89, 113.90, 116.71, 123.61, 127.34, 132.54, 135.16, 137.84, 145.07, 149.46, 173.44

### N-(4-tert-Butylphenyl)anthranilic acid

Yield=87%

White Powder

¹H NMR (400 MHz, CDCl₃)=1.34 (s, 9H), 6.72 (m, 1H), 7.18-7.40 (m, 6H), 8.02(dd, J=1.60 Hz and 1.64 Hz, 1H), 9.27 (bs, 1H); 13C NMR (100 MHz, CDCl3)=31.44, 34.43, 110.00, 113.97, 116.76, 123.11, 126.28, 132.53, 135.14, 139.40, 147.40, 152.21, 170.15

### N-(4-Decylphenyl)anthranilic acid

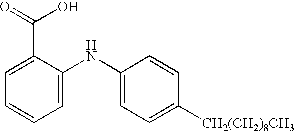

Yield=89%

White Powder

¹H NMR (400 MHz, CDCl₃)=0.88 (t, J=6.84 Hz, 3H), 1.27-1.32 (m, 16H), 2.60 (t, J=7.73 Hz, 2H), 6.72 (quintet, 1H), 7.13-7.18 (m, 5H), 7.30-7.35 (m, 1H), 8.02 (dd, J=1.48 Hz and 1.48 Hz, 1H), 9.25 (bs, 1H); 13C NMR (100 MHz, CDCl3)=14.15, 22.71, 29.36, 29.55, 29.64, 31.60, 31.93, 35.45, 109.90, 113.87, 116.70, 123.65, 129.35, 132.56, 135.19, 139.20, 149.52, 173.60

### N-(4-Methoxyphenyl)anthranilic acid

Yield=94%

Yellow Powder

¹H NMR (400 MHz, CDCl₃)=3.83 (s, 3H), 6.69 (dd, J=7.13 Hz and 7.07 Hz, 1H), 6.94 (t, J=8.99 Hz, 3H), 7.19 (d, J=8.84 Hz, 2H), 7.28-7.32 (m, 1H), 8.01 (dd, J=1.33 Hz and 1.34 Hz, 1H), 9.14 (bs, 1H); 13C NMR (100 MHz, CDCl3)=55.51, 109.32, 113.44, 114.70, 116.28, 126.40, 132.48, 132.94, 135.24, 150.46, 157.00, 173.40

### N-(4-Hexyloxyphenyl)anthranilic acid

Yield=86%

White Powder

¹H NMR (400 MHz, CDCl₃)=0.92 (t, J=7.04 Hz, 3H), 1.33-1.39 (m, 4H), 1.44-1.50 (m, 2H), 1.76-1.83 (m, 2H), 3.97 (t, J=6.58 Hz, 2H), 6.68-6.70 (m, 1H), 6.92 (dd, J=2.16 Hz and 2.12 Hz, 2H), 6.94 (d, J=8.70 Hz, 1H), 7.17 (dd, J=2.18 Hz and 2.11 Hz, 2H), 7.28-7.32 (m, 1H), 8.01 (dd, J=1.58 Hz and 1.58 Hz, 1H), 9.13 (bs, 1H); 13C NMR (100 MHz, CDCl3)=14.05, 22.61, 25.74, 29.27, 31.60, 68.31, 109.26, 113.45, 115.29, 116.21, 126.39, 132.46, 132.70, 135.21, 150.52, 156.59, 173.38

### N-(2-Trifluoromethylphenyl)anthranilic acid

Yield=74%

White Crystal

¹H NMR (400 MHz, CDCl₃)=6.84 (quintet, 1H), 7.16-7.21 (m, 2H), 7.38 (quintet, 1H), 7.50 (t, J=7.55 Hz, 1H), 7.58 (d, J=8.11 Hz, 1H), 7.69 (d, J=7.77 Hz, 1H), 8.09 (dd, J=1.56 Hz and 1.58 Hz, 1H), 9.59 (bs, 1H); 13C NMR (100 MHz, CDCl3)=111.87, 114.65, 118.36, 123.48, 124.04, 127.10, 127.15, 132.49, 132.69, 135.07, 139.10, 147.78, 173.13

### N-(4-Trifluoromethylphenyl)anthranilic acid

Yield=79%

White Powder

¹H NMR (400 MHz, CDCl₃)=6.87-6.91 (m, 1H), 7.33 (d, J=8.40 Hz, 2H), 7.38 (dd, J=0.92 Hz and 1.05 Hz, 1H), 7.42-7.46 (m, 1H), 7.59 (d, J=8.49 Hz, 2H), 8.09 (dd, J=1.52 Hz and 1.58 Hz, 1H), 9.45 (bs, 1H); 13C NMR (100 MHz, CDCl3)=111.91, 114.94, 118.86, 120.70, 126.70, 132.77, 135.31, 143.93, 146.95, 172.90

### N-(4-Nitrophenyl)anthranilic acid

Yield=67%

Yellow Powder

¹H NMR (400 MHz, CDCl₃)=7.00-7.04 (m, 1H), 7.28-7.31 (m, 2H), 7.53-7.54 (m, 2H), 8.13 (d, J=7.89 Hz, 1H), 8.19-8.23 (m, 2H), 9.66 (bs, 1H); 13C NMR (100 MHz, CDCl3)=99.10, 113.61, 116.51, 118.17, 120.63, 125.81, 132.86, 135.28, 141.83, 144.97, 147.27, 170.5

### N-(4-Fluoro-3-trifluoromethylphenyl)anthranilic acid

Yield=62%

White Powder

¹H NMR (400 MHz, CDCl₃)=6.81-6.86 (m, 1H), 7.06-7.08 (m, 1H), 7.18-7.23 (m, 1H), 7.39-7.42 (m, 2H), 7.49 (dd, J=2.68 Hz and 2.63 Hz), 8.07 (dd, J=1.57 Hz and 1.57 Hz, 1H), 9.29 (bs, 1H); 13C NMR (100 MHz, CDCl3)=110.86, 113.61, 117.89, 118.16, 121.96, 128.55, 128.63, 132.81, 135.61, 136.64, 148.40, 173.54

### N-(2,4-Ditrifluoromethylphenyl)anthranilic acid

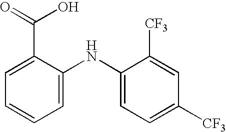

Yield=88%

White Powder

¹H NMR (400 MHz, DMSO)=7.06 (t, J=7.53 Hz, 1H), 7.45 (d, J=8.36 Hz, 1H), 7.51-7.55 (m, 1H), 7.82 (d, J=8.68 Hz, 1H), 7.93 (d, J=9.03 Hz, 1H), 7.97 (s, 1H), 8.00 (d, J=7.96 Hz, 1H), 10.29 (bs, 1H); 13C NMR (100 MHz, DMSO)=116.97, 117.59, 119.72, 121.53, 121.84, 122.64, 123.22, 125.30, 125.92, 131.61, 132.83, 135.13, 144.14, 144.61, 170.78

### Example 2

### Preparation of Anthranilic Acid Derivatives 2 (R₃:NO₂)

### 2-1. Preparation of 2-bromo-5-nitrobenzoic acid methyl ester

2-Bromo-5-nitro benzoic acid methyl ester was prepared according to the Reaction Formula 4 shown below: (yield: 81%):

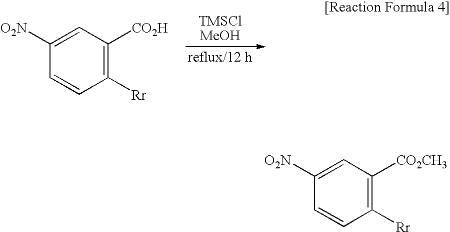

White Crystal

¹H NMR (400 MHz, CDCl₃)=4.00 (s, 3H), 7.88 (d, J=8.76 Hz, 1H), 8.18 (dd, J=2.76 Hz and 2.76 Hz, 1H), 8.66 (d, J=2.74 Hz, 1H) ¹³C NMR (100 MHz, CDCl₃)=53.12, 126.33, 126.61, 129.24, 133.12, 135.71, 146.70, 164.48

2-2. Preparation of N-aryl anthranilic acid

### 5-Nitro-N-(4-trifluoromethylphenyl)anthranilic acid

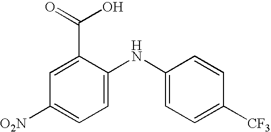

Yield=72%

Yellow Powder

¹H NMR (400 MHz, DMSO)=7.39 (d, J=9.40 Hz, 1H), 7.58 (d, J=8.40 Hz, 2H), 7.79 (d, J=8.56 Hz, 2H), 8.23 (dd, J=2.84 Hz and 2.80 Hz, 1H), 8.74 (d, J=2.84 Hz, 1H), 10.56 (bs, 1H); 13C NMR (100 MHz, CDCl3)=113.86, 115.29, 123.82, 127.85, 129.21, 130.23, 138.68, 143.46, 151.81, 169.35

The below chemicals were prepared according to the above Example 2-1 and 2-2:

### 5-Nitro-N-(4-nitrophenyl)anthranilic acid

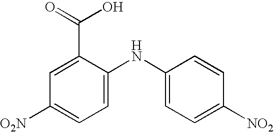

Yield=53%

Orange Solid

¹H NMR (400 MHz, DMSO)=7.48 (d, J=9.04 Hz, 2H), 7.61 (d, J=9.14 Hz, 1H), 8.19 (dd, J=2.85 Hz and 2.92 Hz, 1H), 8.27 (d, J=8.98 Hz, 2H), 8.88 (d, J=2.85 Hz, 1H); 13C NMR (100 MHz, CDCl3)=114.79, 119.43, 124.63, 126.76, 127.23, 128.65, 139.47, 141.79, 147.93, 149.96, 169.04

### Example 3

### Measurement of Physiological Activity by Using Two-Electrode Voltage Clamp Method

Eggs (ovum cells) of Xenopus Laevis were collected by surgery and cultivated in Barth's solution [88 mM NaCl, 1 mM KCl, 2.4 mM NaHCO3, 10 mM HEPES, 0.82 mM MGSO4, 0.33 mM Ca(NO3)2, 0.91 mM CaCl2 (the above reagents were purchased from Sigma); 10 units/ml penicillin, streptomycin (purchased from Gibco)] inside a culture chamber at 18° C. The prepared eggs were treated with 10 μM ionomycin (Sigma) for 30 min. The above eggs treated with ionomycin were washed with the recording solution (96 mM NaCl, 2 mM KCL, 10 mM HEPES, 3 mM NaOH, 2 mM MgCl2, 0.5 mM EGTA, Sigma), subsequently treated with 1 μM thapsigargin (Sigma) for 90 min, and washed with the above recording solution to prepare the eggs for recording.

An intracellular solution was prepared by adding 0.5 mM EGTA and 1 μM Chelerythrine (Sigma) into a 1 M KCl solution, which was filled in glass microelectrodes. One of the above prepared eggs was placed on the chamber and two electrodes were inserted into opposite sides of the egg while the recording solution was poured. Current was measured by performing two-electrode voltage clamp recording according to a protocol suitable for the purpose of the experiment.

FIG. 1 shows the changes in the current according to the thapsigargin treatment in Xenopus Laevis egg, wherein FIG. 1a shows the current induced in a dose-dependent manner from extracellular calcium ions in eggs treated with ionomycin, and FIG. 1b shows EC₅₀ and dose response of calcium ion, i.e. % peak current value according to the amount of treated calcium ions, obtained in the above FIG. 1a. FIG. 1c shows the current recording obtained after the treatment of 10 μM ionomycin for 30 min. without the treatment of thapsigargin, where (1) shows a fast peak and a slow steady state during the application of 5 mM calcium ion for 90 seconds, (2) shows a slight decrease in the fast peak by secondary application of calcium ions for 5 seconds to show the fast peak induced by the calcium ion, (3) shows that the slow component induced by calcium ions may be blocked by substituting calcium ions with barium ions, and (4) shows that barium ions do not induce a fast peak. FIG. 1d shows the current obtained after the treatment of 10 μM ionomycin for 30 min. and subsequent treatment of 1 μM thapsigargin for 90 min., where (1) shows a fast peak and a slow steady state during the application of 5 mM calcium ion for 90 seconds, (2) shows a fast peak with a similar amplitude to the first peak by secondary application of calcium ions for 5 seconds to show the fast peak induced by the calcium ions, (3) shows that the slow component induced by calcium ions may be blocked by substituting calcium ions with barium ions, and (4) shows that barium ions do not induce a fast peak.

FIGS. 1e˜1i show graphs comparing the current according to the treatment conditions of Chelerythrine (CHE) and thapsigargin (TG), where CHE+ means the case when Chelerythrine was added to the intracellular solution and TG+ means that the eggs pretreated with ionomycin were treated with thapsigargin. FIG. 1e shows the amplitude of a fast peak, FIG. 1f shows the amplitude of a slow steady state component, FIG. 1g summarizes the results under the above 1c(2) and 1d(2) conditions, FIG. 1h summarizes the results under the above 1c(3) and 1d(3) conditions, and FIG. 1i summarizes the results under the above 1c(4) and 1d(4) conditions,

In the above Figures, n is the number of eggs, error bars mean SEMs, and * shows statistically meaningful differences by a two-tailed t-test.

From FIGS. 1a˜1i as above, it may be confirmed that a fast peak may be induced by a secondary application of the calcium ions by recording the changes of the current in the ionomycin-treated eggs by treating them with thapsigargin and by using the intracellular solution added with chelerythrine. From the above experimental procedures, it may also be confirmed that the degree of decrease of the secondary peak obtained from the channel blocking agent candidate material as compared to the first peak induced by calcium ions.

In order to activate a Ca²⁺-dependent chloride channel, 5 mM Ca²⁺ was treated first to the above eggs for 5 seconds. After being washed with the above recording solution for 50 seconds, and pretreated with the chemicals in the below Table 1 (treated in the order of 1, 3, 10, 30, 100, 300 mM) for 15 seconds, the eggs were treated with 5 mM Ca²⁺ and the above chemicals (same amount as above) to measure the inhibitory concentrations of each chemical by comparing the current measured with 5 mM Ca²⁺.

The IC₅₀ values obtained from the above experiment for calcium ion-activated chloride channels of the conventionally used inhibitors and anthranilic acid derivatives according to the present invention are listed in Table 1.

TABLE 1

| chemical compound | IC50 | n |
|---|---|---|
| DIDS | 10.7 | 6 |
| NPPB(5-nitro-2-(3-phenylpropylamino)benzoic acid) | 32.3 | 6 |
| Indomethacin | LP | 8 |
| 9-AC(9-anthracene carboxylic acid) | 94.3 | 5 |
| Niflumic acid | 37.3 | 7 |
| Flufenamic acid | 35.4 | 6 |
| Mefenamic acid | 44.5 | 6 |
| N-Phenylanthranilic acid | 88.1 | 6 |
| 5-Nitro-N-phenylanthranilic acid | 42.5 | 8 |
| N-(4-Nitrophenyl)anthranilic acid | 17.8 | 7 |
| N-(3-Nitrophenyl)anthranilic acid | 32.1 | 7 |
| N-(4-Trifluoromethylphenyl)anthranilic acid | 6.0 | 6 |
| N-(2-Trifluoromethylphenyl)anthranilic acid | 29.5 | 6 |
| 5-Nitro-N-(4-nitrophenyl)anthranilic acid | 15.4 | 5 |
| N-(4-Fluorophenyl)anthranilic acid | 63.1 | 6 |
| N-(4-Chlorophenyl)anthranilic acid | 11.3 | 6 |
| N-(4-Methylphenyl)anthranilic acid | 55.3 | 7 |
| N-(4-Isopropylphenyl)anthranilic acid | 17.0 | 6 |
| N-(4-tert-Butylphenyl)anthranilic acid | 22.9 | 7 |
| N-(4-Fluoro-3-trifluoromethylphenyl)anthranilic acid | 14.7 | 6 |
| N-(4-Methoxyphenyl)anthranilic acid | 102.3 | 5 |

DIDS: disodium 4,4′-diisothiocyano-2,2′-stilbenedisulphonic acid
LP: Low Potency IC₅₀ > 200 μM
n: number of eggs

In the above Table 1, IC₅₀ values from DIDS to 5-nitro-N-phenylanthranilic acid are results for the conventionally used inhibitors, and IC₅₀ values from N-(4-nitrophenyl)anthranilic acid to N-(4-methoxylphenyl)anthranilic acid are results from the chemicals according to the present invention.

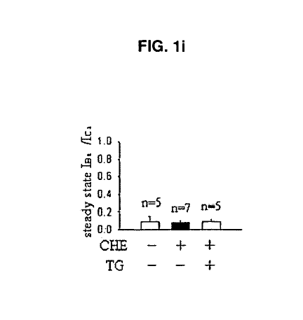

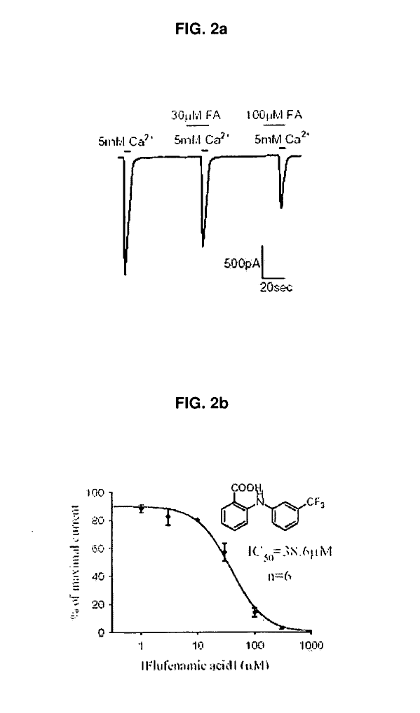

FIGS. 2a˜2c show the effect of known blocking agents of the chloride channel activated by calcium ions, wherein 2a shows the current changes in the chloride channel activated by calcium ions before and during the application of flufenamic acid (FA), 2b shows the dose response relationship in the inhibition of the chloride channel activated by calcium ions by flufenamic acid, and 2c summarizes the IC₅₀ values of commercialized blocking agents for the chloride channel activated by calcium ions. In the above figure, n is the number of eggs and the error bar means SEMs.

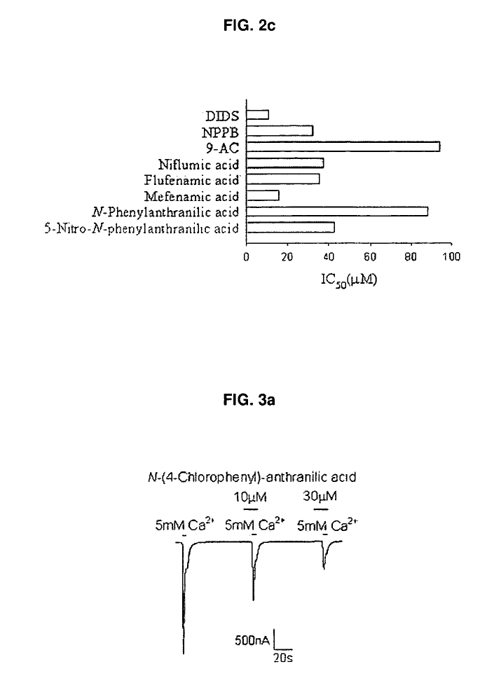

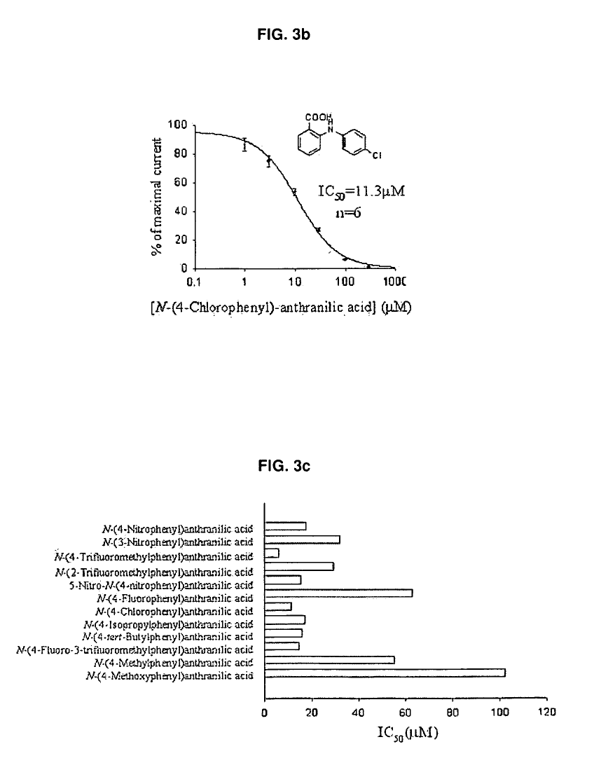

FIGS. 3a˜3c show the effect of anthranilic acid derivatives according to the present invention of the chloride channel activated by calcium ions, wherein 3a shows the current changes in the chloride channel activated by calcium ions before and during the application of N-(4-chlorophenyl)-anthranilic acid, 3b shows the dose response relationship in the inhibition of a chloride channel activated by calcium ions by N-(4-chlorophenyl)-anthranilic acid (0, 10 μM, 30 μM), and 3c summarizes the IC₅₀ values of anthranilic acid derivatives for the chloride channel activated by calcium ions. In the above figure, n is the number of eggs and the error bar means SEMs.

As may be seen in FIG. 2, FIG. 3, and Table 1, anthranilic acid derivatives according to the present invention have the IC₅₀ values for the calcium ion activation chloride channel that are similar to or lower than those of known inhibitors, confirming the excellent calcium ion activated chloride channel inhibitory activity of the anthranilic acid derivatives according to the present invention. Moreover, N-(4-trifluoromethylphenyl)anthranilic acid, N-(4-chlorophenyl)anthranilic acid, N-(4-fluoro-3-trifluoromethylphenyl)anthranilic acid, 5-nitro-N-(4-nitrophenyl)anthranilic acid, N-(4-nitrophenyl)anthranilic acid, N-(4-tert-butylphenyl)anthranilic acid, N-(2-trifluoromethylphenyl)anthranilic acid, and N-(3-nitrophenyl)anthranilic acid have notably high inhibitory activity of calcium activated chloride channel current when compared to the conventional anthranilic acid derivatives, flufenamic acid and mefenamic acid known as chloride channel blocking agents.

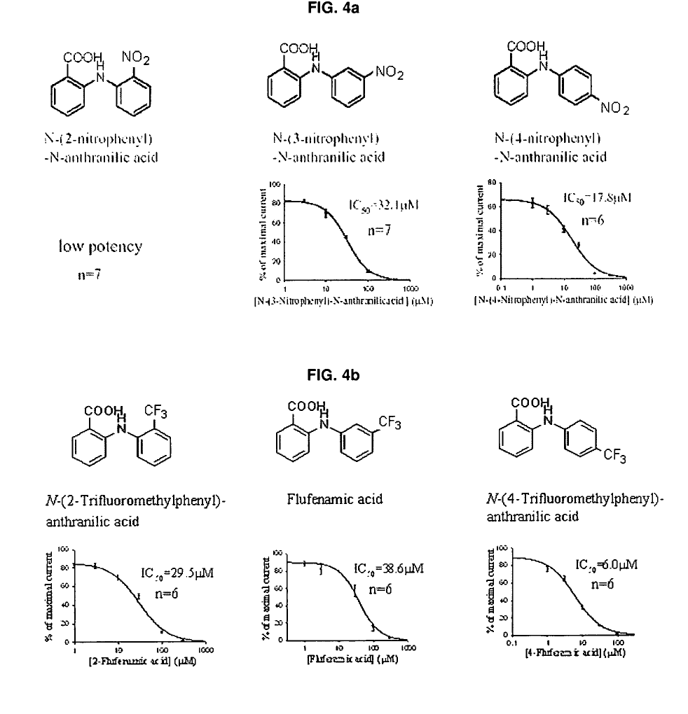

In the meantime, FIG. 4 shows the effect of a calcium channel blocking agent to the chloride channel activated by calcium ions according to the position of functional groups in the phenyl ring. FIG. 4a shows the IC₅₀ values when the nitro group bonding to the benzene ring of the anthranilic acid derivatives in ortho, meta, and para positions, and inhibitory activity is higher when the nitro group is positioned in meta or para positions than in the ortho position. Also, FIG. 4b shows IC₅₀ values when the trifluoromethyl group (—CF₃) bonding to the benzene ring of the anthranilic acid derivatives in ortho, meta, and para positions, and inhibitory activity is higher when the trifluoromethyl group is positioned in ortho or para positions when compared to the flufenamic acid, where the trifluoromethyl group is bonded in the meta position of the anthranilic acid derivatives. From the results of FIGS. 4a and 4b, inhibitory activity is prominently higher when a nitro group (—NO₂) or trifluoromethyl group (—CF₃) is bonded at the para positions of the benzene ring.

While this invention has been described in connection with what is presently considered to be practical exemplary embodiments, it is to be understood that the invention is not limited to the disclosed embodiments, but, on the contrary, is intended to cover various modifications and equivalent arrangements included within the spirit and scope of the appended claims.

What is claimed is:

1. A compound represented by Chemical Formula I: 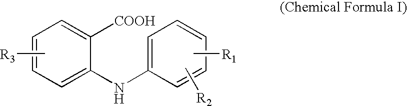
wherein
R₁ and R₂, respectively, are independently selected from the group consisting of a hydrogen atom, a halogen atom, a linear, branched, or cyclic alkyl group having 5 to 16 carbon atoms, a linear or branched substituted alkyl group having 1 to 16 carbon atoms, a substituted or non-substituted aryl group having 6 to 12 carbon atoms, an alkoxy group having 2 to 8 carbon atoms, a substituted alkoxy group having 1 to 8 carbon atoms, and a nitro group, and R₁ and R₂ are not hydrogen at the same time,
R₃ is a hydrogen atom or a nitro group, and
when R₃ is a hydrogen atom, if one of R₁ and R₂ is 3-trifluoromethyl, then the other is not a hydrogen atom; if one of R₁ and R₂ is 2-trifluoromethyl, then the other is not 3-trifluoromethyl; if one of R₁ and R₂ is 4-fluoro, 4-chloro-, or 4-bromo, then the other is not a hydrogen atom; if one of R₁ and R₂ is 2-chloro, then the other is not 3-chloro or 4-chloro; and if one of R₁ and R₂ is 3-chloro, then the other is not 4-chloro or 5-chloro.

2. The compound according to claim 1, wherein
the substituted alkyl or alkoxy group is a compound in which one or more hydrogen atoms in an alkyl or alkoxy group are independently substituted with one or more selected from the group consisting of halogen atoms and a phenyl group, and
the substituted aryl group is a compound in which one or more hydrogen atoms in an aryl group are independently substituted with one or more selected from the group consisting of a halogen atom and an alkyl group having 1 to 6 carbon atoms.

3. The compound according to claim 1, wherein the R₁ and R₂, respectively, are independently selected from the group consisting of hydrogen, a pentyl group, a hexyl group, a heptyl group, an octyl group, a decyl group, a dodecyl group, a tetradecyl group, a hexadecyl group, a cyclohexyl group, a phenyl group, a benzyl group, a racemic-alpha methyl-benzyl group, a chiral-alpha methyl-benzyl group, a trifluoromethyl group, a methoxyl group, an ethoxyl group, a butyloxy group, a pentyloxy group, a hexyloxy group, a trifluoromethyloxy group, and a nitro group, or the R₁ and R₂ are the same or different halogen atoms.

4. The compound according to claim 1, which is selected from the group consisting of decylphenyl anthranilic acid, hexaoxyphenyl anthranilic acid, trifluoromethylphenyl anthranilic acid, nitrophenyl anthranilic acid, 4-fluoro-3-trifluoromethylphenyl anthranilic acid, 2,4-ditrifluoromethylphenyl anthranilic acid, 4-trifluoromethylanilino-5-nitrobenzoic acid, and 4-nitroanilino-5-nitrobenzoic acid.

5. A method of preparing a compound represented by Chemical Formula I, comprising the steps of:
reacting a compound represented by Chemical Formula 1 with chlorotrimethylsilane in the presence of methanol or ethanol, or esterifying by using methanol or ethanol in the presence of an acid catalyst, to prepare a compound represented by Chemical Formula 2;
reacting the obtained compound represented by Chemical Formula 2 with an aniline compound represented by Chemical Formula 3, to prepare a compound represented by Chemical Formula 4; and
reacting the compound represented by Chemical Formula 4 with one or more selected from the group consisting of alkali metal hydroxides and alkali earth metal hydroxides, to obtain the compound represented by Chemical Formula I, 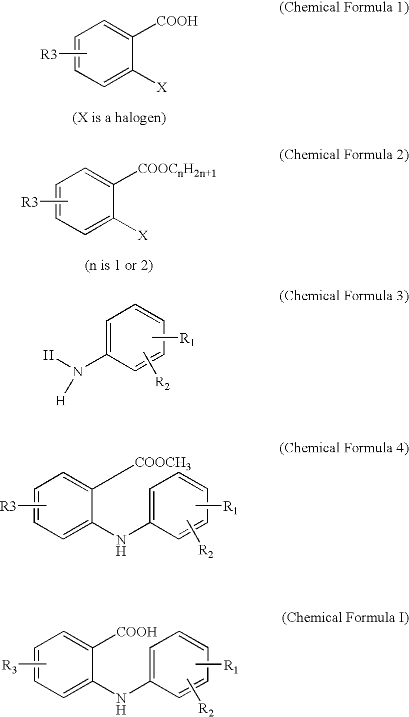
wherein
R₁ and R₂, respectively, are independently selected from the group consisting of a hydrogen atom, a halogen atom, a linear, branched, or cyclic alkyl group having 5 to 16 carbon atoms, a linear or branched substituted alkyl group having 1 to 16 carbon atoms, a substituted or non-substituted aryl group having 6 to 12 carbon atoms, an alkoxy group having 2 to 8 carbon atoms, a substituted alkoxy group having 1 to 8 carbon atoms, and a nitro group, and R₁ and R₂ are not hydrogen at the same time,
R₃ is a hydrogen atom or a nitro group, and
when R₃ is a hydrogen atom, if one of R₁ and R₂ is 3-trifluoromethyl then the other is not a hydrogen atom, if one of R₁ and R₂ is 2-trifluoromethyl then the other is not 3-trifluoromethyl, if one of R₁ and R₂ is 4-fluoro, 4-chloro-, or 4-bromo, then the other is not a hydrogen atom, if one of R₁ and R₂ is 2-chloro then the other is not 3-chloro or 4-chloro, and if one of R₁ and R₂ is 3-chloro then the other is not 4-chloro or 5-chloro.

6. A composition for inhibiting a chloride channel comprising a compound represented by Chemical Formula I-1 or its pharmacologically acceptable salts as an active ingredient: 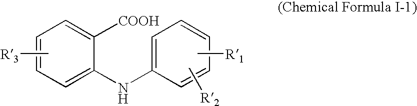
wherein
R′₁, and R′₂ are independently selected from the group consisting of a hydrogen atom, a halogen atom, a linear, branched, or cyclic substituted or non-substituted alkyl group having 1 to 16 carbon atoms, a substituted or non-substituted aryl group having 6 to 12 carbon atoms, a substituted or non-substituted alkoxy group having 1 to 8 carbon atoms, and a nitro group,
R′₁ and R′₂ are not hydrogen atoms at the same time,
R′₃ is a hydrogen atom or a nitro group, and
when R′₃ is a hydrogen atom, if one of R′₁ and R′₂ is 3-trifluoromethyl then the other is not a hydrogen atom, and if one of R′₁ and R′₂ is 2-trifluoromethyl then the other is not 3-trifluoromethyl.

7. The composition according to claim 6, wherein;
the substituted alkyl or alkoxy group is a compound in which one or more hydrogen atoms in an alkyl or alkoxy group are substituted independently with one or more selected from the group consisting of halogen atoms and a phenyl group; and
the substituted aryl group is a compound in which one or more hydrogen atoms in the aryl group are independently substituted with one or more selected from the group consisting of halogen atoms and an alkyl group having 1 to 6 carbon atoms.

8. The composition according to claim 6, wherein the compound is one or more selected from the group consisting of N-(4-trifluoromethylphenyl)anthranilic acid, N-(4-chlorophenyl)anthranilic acid, N-(4-fluoro-3-trifluoromethylphenyl)anthranilic acid, 5-nitro-N-(4-nitrophenyl)anthranilic acid, N-(4-nitrophenyl)anthranilic acid, N-(4-tert-butylphenyl)anthranilic acid, N-(2-trifluoromethylphenyl)anthranilic acid, and N-(3-nitrophenyl)anthranilic acid.

9. The composition according to claim 6, wherein the content of the compound is 0.01 wt % to 100 wt %.

10. The composition according to claim 6, which is used for preventing or treating one or more diseases selected from the group consisting of fibroma, inflammation, and dystrophy.

11. A method of determining of the inhibition of chloride channel by using a two-electrode voltage clamp method, comprising the steps of:
preparing a sample;
treating the sample with calcium;
treating the sample with ionomycin;
treating the ionomycin-treated sample with thapsigargin;
contacting two microelectrodes filled with intracellular solution containing chelerythrine with the ionomycin and thapsigargin treated sample; and
measuring the current generated from the electrodes.

12. A method of screening a chloride channel blocking agent by using a two-electrode voltage clamp method, comprising the steps of:
treating samples with calcium;
treating some of the samples with a candidate material, to prepare candidate material treated samples and untreated samples;
treating the candidate material treated samples and untreated samples with ionomycin and then thapsigargin;
contacting two microelectrodes filled with intracellular solution containing chelerythrine with the samples; and
determining the candidate material as a chloride channel blocking agent when the induced current of the candidate material treated samples is decreased or not changed compared with that of untreated samples.
